(19) United States
(12) Patent Application Publication
(10) Pub. No.: US 2014/0308426 A1
(43) Pub. Date: Oct. 16, 2014

(54) WATER-SOLUBLE SOYBEAN POLYSACCHARIDES AND MANUFACTURING PROCESS THEREFOR

(71) Applicant: Fuji Oil Company Limited, Osaka-shi (JP)

(72) Inventors: Yasuhiko Yoshida, Ibaraki (JP); Minami Sato, Ibaraki (JP); Akihiro Nakamura, Ibaraki (JP)

(73) Assignee: Fuji Oil Company Limited, Osaka-shi (JP)

(21) Appl. No.: 14/352,453

(22) PCT Filed: Oct. 18, 2012
(86) PCT No.: PCT/JP2012/076895

§ 371 Date: Apr. 17, 2014

(30) Foreign Application Priority Data
Oct. 20, 2011 (JP) 2011-230413

(51) Int. Cl. C08B 37/00; A23D 7/00

(52) U.S. Cl. CPC C08B 37/00; A23D 7/00
USPC 426/602; 536/123.1

(57) ABSTRACT

[Problem] The purpose of the present invention is to provide a natural polymeric emulsifier improved in so-called “emulsifying power”, said natural polymeric emulsifier being capable of forming, even by the addition thereof in a small amount, an oil-in-water emulsion which has a small emulsified particle diameter and a uniform particle size distribution and which rarely suffers from the aggregation or coalescence of emulsified particles even when has undergone heating, long-term storage, aging or the like.

[Solution] The purpose can be achieved by using water-soluble soybean polysaccharides which have a methyl ester content of 30% or below relative to the uronic acids and an acetate content of 1 wt % or higher in terms of free acetic acid and relative to the water-soluble soybean polysaccharides. The water-soluble soybean polysaccharides can be manufactured by subjecting raw material water-soluble soybean polysaccharides to methyl ester removal treatment and thereafter acetate-forming esterification treatment.

### TECHNICAL FIELD

The present invention relates to a modified water-soluble soybean polysaccharide. Specifically, the present invention relates to a water-soluble soybean polysaccharide having superior emulsifying capacity and emulsion-stabilizing property compared to those of conventional water-soluble soybean polysaccharide. More particularly, the present invention relates to a water-soluble soybean polysaccharide obtained by a method of the present invention having low methyl ester content and appropriate acetic acid ester content which are suitable for high emulsifying capacity and emulsion-stabilizing property, an emulsifier including the water-soluble soybean polysaccharide as an active ingredient, an oil-in-water emulsion including the emulsifier, and a use of the water-soluble soybean polysaccharide for preparing an emulsifier and an oil-in-water emulsion.

### BACKGROUND ART

A polymer emulsifier is used for emulsified flavor, which is typically used for flavoring a beverage, because it allows keeping emulsion without breaking the emulsified state even if the emulsion is highly diluted, and it has good stability to heat and stability under acidic condition. Examples of polymer emulsifier includes gums such as gum Arabic, natural products such as casein and synthetic compounds such as acrylate and polyvinyl alcohol, as well as cross-linked starch octenylsuccinate which is amphiphilic for providing emulsifying capacity.

Gum Arabic stabilizes an oil-in-water emulsion (O/W type) by function of sugar chain in the gum Arabic as hydrophilic group and function of polypeptide bound to the sugar chain as hydrophobic group (Non-Patent Document 1). Gum Arabic is used as a good emulsifier for emulsified flavor because an emulsion prepared by using gum Arabic forms thick hydrophilic layer of sugar chain in an interface of an oil drop, and gum Arabic is less likely to separate in the oil drop even when the emulsion is highly diluted. However, it is necessary to use at high concentration, typically to add 12 wt % or more of gum Arabic when the oil phase is 20 wt % (i.e., 0.6 parts by weight or more relative to 1 part of oil). In addition, supply quantity of gum Arabic varies with climate of the producer country, and market price of gum Arabic is not constant. Therefore, a polymer emulsifier which can be constantly supplied is desired (Non-Patent Document 2).

A water-soluble modified gum Arabic having 0.9 million or more of weight-average molecular weight obtained by heating gum Arabic derived from Acacia Senegal has good emulsifying capacity and emulsion-stabilizing property. However, homogenization with homogenizer is necessary in order to prepare an emulsion having less than 1 μm of emulsion particle diameter (Patent Document 1).

In addition, although a synthetic acrylate or polyvinyl alcohol etc. has good emulsifying capacity, its application is often limited due to high viscosity.

Further, there are various modified starches such as etherified starch, esterified starch, cross-linked starch and grafted starch. Among them, one of the esterified starch, starch octenylsuccinate has good emulsifying capacity, but has extremely low emulsion-stabilizing property in the system of highly diluted, and is not applicable for long storage. In addition, although starch acetate in which ester acetate is bound to starch is commercially available, it has no emulsifying capacity and it is used for providing a resistance to retrograde and viscosity stability.

Water-soluble soybean polysaccharide obtained by carrying out demethoxyl treatment, which provides high dispersion stability of protein particles under acidic condition and which has relatively low viscosity, is reported (Patent Document 2). However, a lot of additive amount is necessary in order to obtain strong emulsifying capacity. Therefore, further improvement is desired.

### PRIOR ART DOCUMENTS

### Patent Documents

Patent Document 1: JP 2006-522202 A
Patent Document 2: JP H05-262802 A

### Non-Patent Documents

Non-Patent Document 1: Glicksman, Food Hydrocolloids. Boca Raton, Fla.: CRC Press. P 7-30, 1983
Non-Patent Document 2: Food Polysaccharides—knowledge of emulsifying, thickening and gelatinization—, Saiwai Shobo Co. Ltd., p 77-84

### DISCLOSURE OF INVENTION

### Problems to be Solved by the Invention

A conventional water-soluble soybean polysaccharide has required a lot of additive amount in order to provide high emulsifying capacity and emulsion-stabilizing property, and thus, it has caused a problem that viscosity is increased. An object of the present invention is to provide natural polymer emulsifier having improved so-called “emulsifying capacity”, that is, emulsion particle diameter of oil-in-water emulsion is small, particle size distribution in the emulsion is uniform, coagulation or incorporation of emulsion particles by heating, long storage, aging or the like are suppressed, even if small amount of emulsifier is added.

A conventional emulsified flavor including gum Arabic has achieved insufficient function of suppressing oil float and maintaining turbidity when it is added to alcohol beverage. An object of the present invention is also to provide function of stabilizing emulsion particle with smaller additive amount than that of conventional gum Arabic to water-soluble soybean polysaccharide, in addition to the above functions. In addition, an object of the present invention is also to provide function of emulsifier that can make particle diameter small for obtaining stability of emulsion with only gentle stirring without homogenization treatment by using high pressure homogenizer to water-soluble soybean polysaccharide.

### Means for Solving the Problems

The present inventors have intensively studied in order to solve the problems. As a result, the present inventors have found out that strong emulsifying capacity can be added to a water-soluble soybean polysaccharide by subjecting the water-soluble soybean polysaccharide to de-methylesterification treatment followed by acetic acid esterification, different from the case of esterifying starch. The present invention has been completed on the basis of these findings.

That is, the present invention relates to:

(1) a water-soluble soybean polysaccharide comprising 30% or less of methyl ester content relative to uronic acid and 1 wt % or more of acetic acid ester content relative to the water-soluble soybean polysaccharide in terms of free acetic acid;

(2) a process for producing the water-soluble soybean polysaccharide according to (1), comprising subjecting acetic acid esterification treatment to a water-soluble soybean polysaccharide which is subjected to de-methylesterification treatment;

(3) an emulsifier comprising the water-soluble soybean polysaccharide according to (1) as an active ingredient;

(4) an oil-in-water emulsion, which is obtained by using the emulsifier according to (3) to stabilizing dispersion of hydrophobic material in hydrophilic solvent;

(5) the oil-in-water emulsion according to (4), wherein 0.3 part by weight or more and 7 parts by weight or less of the emulsifier according to (3) is used to 1 part by weight of hydrophobic material;

(6) a use of the water-soluble soybean polysaccharide according to (1) for preparing an emulsifier; and

(7) a use of the water-soluble soybean polysaccharide according to (1) for preparing an emulsion.

### Effect of the Invention

A water-soluble soybean polysaccharide of the present invention can provide emulsifying capacity and emulsion-stabilizing property, low viscosity and small emulsion particle diameter by lower amount than conventional water-soluble soybean polysaccharide, and has less effect on taste, and therefore, can enlarge the range of formulation of final product.

### Mode for Carrying Out the Invention

A water-soluble soybean polysaccharide of the present invention meets that methyl ester content relative to uronic acid is 30% or less and that acetic acid ester content relative to the water-soluble soybean polysaccharide is 1 wt % or more in terms of free acetic acid. In addition, the water-soluble soybean polysaccharide can be obtained by subjecting a water-soluble soybean polysaccharide to de-methylesterification treatment followed by acetic acid esterification.

Hereinafter, the present invention will be concretely explained.

### (Water-Soluble Soybean Polysaccharide)

As a water-soluble soybean polysaccharide used as a raw material in the present invention, a soybean polysaccharide obtained by various methods can be used. For example, a water-soluble soybean polysaccharide described in JP 2599477 B can be applied. Hereinafter, an example of the production of the water-soluble soybean polysaccharide will be explained.

### (Raw Material of Water-Soluble Soybean Polysaccharide)

Soybean is suitable for a raw material of water-soluble soybean polysaccharide. In particular, the raw material of water-soluble soybean polysaccharide is preferably cotyledon of soybean, more preferably, so-called “okara” obtained as a by-product of tofu or soybean protein isolate because okara includes abundant polysaccharide. In addition, a concentration of polysaccharide can be increased by subjecting these raw materials to proteolytic enzyme treatment or alkaline solution treatment in advance, and then extracting and removing residual protein from the raw material. Okara as by-product of tofu is preferably used as raw material because water-soluble fraction is already removed. In addition, okara as by-product of soybean protein isolate is more preferably used as raw material because both water-soluble fraction and lipophilic constituent are already removed.

### (Production Process of Water-Soluble Soybean Polysaccharide)

Extraction with heating from the above-mentioned raw material is carried out. Extraction of water-soluable soybean polysaccharide is carried out as high temperature extraction in aqueous system under weak acidic condition close to an isoelectric point of protein. Extraction temperature is preferably 100° C. or more because high extraction efficiency can be achieved. When the temperature is less than 100° C., it is not preferable on practical side, for example, it takes time for the extraction, yield may be reduced, and water-soluble soybean polysaccharide having insufficient physical property may be obtained. In addition, upper limit of extraction temperature is not particularly limited, but extremely high temperature is not preferable because side reaction may be caused and obtained product tends to be colored. Typically, it can be carried out at 180° C. or less, preferably 150° C. or less.

After the extraction, solid matter and extraction liquid are separated by well-known method such as filtration and centrifugation. In this case, when reaction liquid after heating is neutralized to a range from neutral to weak acidic, the obtained product may be suitable for use for food. The extract filtrate is, directly or after drying, subjected to the following de-methylesterification reaction and acetic acid esterification.

### (De-Methylesterification Treatment)

The water-soluble soybean polysaccharide obtained by the above exemplified process or other process is used to prepare a water-soluble soybean polysaccharide of the present invention.

A water-soluble soybean polysaccharide in which methyl ester content relative to uronic acid is 30% or less is essential for the present invention. That is, methyl esterified carboxyl group in uronic acid which is constituent sugar of water-soluble soybean polysaccharide is necessary to be subjected to de-methylesterification. As a method for de-methylesterification, acid, alkali or enzyme can be used. In view of ease and costs, acid or alkali is preferably used. In view of efficiency, alkali is most preferably used. This de-methylesterification can be carried out before extracting water-soluble soybean polysaccharide, but preferably, it is carried out after extracting.

A pH of extract of the water-soluble soybean polysaccharide or an aqueous solution of the dried water-soluble soybean polysaccharide is adjusted to 9-14, preferably 11-13 with alkali when de-methylesterification is carried out after extracting. The alkali to be used includes, for example, sodium hydroxide, potassium hydroxide, calcium hydroxide, ammonia and the like. After adjusting pH, this solution is heated to higher than ordinary temperature, preferably 40° C. or higher. The higher the temperature and pH is, the higher the effect of this step is. However, the higher pH and heating temperature tend to cause coloring.

Then, solid-liquid separation is carried out by centrifugation, dewatering with centrifugation, press filtering, or the like. The solid-liquid separation can be carried out either under the above described alkaline condition or under weak acidic to neutral pH. In the latter case, protein content in a water-soluble polysaccharide composition can be reduced because isoelectric point precipitation of protein which is extracted from raw material with alkali occurs. In the water-soluble soybean polysaccharide with de-methylesterification treatment, methyl ester content relative to uronic acid is essentially 30% or less, preferably 20% or less. If it is more than 30%, emulsifying capacity may be reduced.

### (Acetic Acid Esterification Treatment)

Acetic acid esterification is carried out by adding a material forming acetic acid ester with hydroxyl group, such as anhydrous acetic acid, vinyl acetate, glacial acetic acid, acetyl chloride and ketene, to an aqueous solution of a water-soluble soybean polysaccharide or mixed solution of the aqueous solution and polar organic solvent such as alcohol and acetone. Among them, anhydrous acetic acid and vinyl acetate are preferable in the viewpoint of safety of the production. Although glacial acetic acid is safety, it may be weakly-reactive. Acetyl chloride has danger of reacting intensely with water. Ketene has a problem of handling difficulty because it is toxic gas.

The reaction is carried out with maintaining neutral to alkaline condition while stirring. As an alkaline agent to be added, for example, hydroxide of alkali metal such as potassium hydroxide, sodium hydroxide and lithium hydroxide; carbonate of alkali metal such as potassium carbonate, sodium carbonate, lithium carbonate and sodium hydrogen carbonate; organic acid salt of alkali metal such as sodium citrate and sodium sodium oxalate; inorganic acid salt of alkali metal such as trisodium phosphate; and hydroxide of bivalent metal such as calcium hydroxide and magnesium hydroxide, and ammonia can be used.

Since pH of the reaction solution is reduced during the reaction, the above alkaline agent is added in a form of solid or solution in order to maintain pH. The reaction pH is preferably pH 6 to 10, more preferably pH 7 to 9. When pH is lower than 6, the acetic acid esterification of water-soluble soybean polysaccharide may be insufficient. When pH is higher than 10, the acetic acid esterification of water-soluble soybean polysaccharide may be insufficient due to desorption of water-soluble soybean polysaccharide. In addition, the reaction temperature can be adjusted to a temperature of which anhydrous acetic acid or vinyl acetate is dissolve in the reaction solution, for example, preferably 0° C. or higher and 60° C. or lower, more preferably 10° C. or higher and 50° C. or lower. When the temperature is higher than 60° C., hydrolysis of anhydrous acetic acid is accelerated and anhydrous acetic acid may be hydrolyzed to acetic acid before reacting with the water-soluble soybean polysaccharide. On the other hand, a temperature less than 0° C. is unrealistic because such a temperature is lower than the melting point of water.

In a water-soluble soybean polysaccharide of the present invention, an acetic acid ester is bound to polysaccharide. Maximum number of introduced acetic acid ester is determined based on the number of hydroxyl group of the polysaccharide. However, desired effect can be achieved even if acetic acid ester is not introduced into all hydroxyl groups. More specifically, acetic acid ester content relative to the water-soluble soybean polysaccharide is necessary to 1 wt % or more, preferably 2 wt % or more, more preferably 5 wt % or more in terms of free acetic acid. A water-soluble soybean polysaccharide having such a composition is suitable for an objective physical property. In addition, the obtained function is not increased even if it exceeds 10 wt %. Therefore, it is preferably less than 10 wt.

### (Purification Treatment)

If necessary, the obtained reaction solution including the water-soluble soybean polysaccharide is subjected to purification treatment as is or after neutralization. Examples of method for the purification treatment include reprecipitation method using polar organic solvent such as methanol, ethanol, isopropanol and acetone; activated carbon treatment; resin adsorption treatment; ultrafiltration method; reverse osmosis method; gel filtration method; dialysis method; ion-exchange resin method; electrodialysis method and ion-exchange membrane method, and combination thereof. Especially, using reprecipitation method using polar organic solvent, ultrafiltration method, reverse osmosis method, gel filtration method or dialysis method is advantageous because various low-molecular substances can also be removed. When the purification treatment is carried out with demineralization, it is desirable to carry out the treatment so that ash content of the polysaccharide after the treatment is 15 wt % or less, preferably 5 to 10 wt %.

When deproteination of raw material is not carried out prior to the above treatment, protein precipitates in the vicinity of isoelectric point during the neutralization. Therefore, it is preferable to remove this precipitation by filtration or centrifugation. Dried water-soluble soybean polysaccharide can be obtained by drying the obtained product by spray drying, freeze-drying, drum drying or the like after the above purification treatment with or without concentration.

### (Quantitation Method of Methyl Esterification Degree)

Methyl esterification degree is determined in accordance with a standard method of determining methyl esterification degree of pectin. That is, it can be calculated from titer obtained by using sample solutions before and after de-methylesterification with alkali according to the following formula:

Methyl esterification degree (DE)=V2/(V1−V2)×100

In this formula, V1 is titer of 0.1N sodium hydroxide (ml) in the titration of a sample solution prepared by adding isopropyl alcohol with hydrochloric acid to starting sample to make uronic acid in the sample free, and then washing with isopropyl alcohol, and then removing hydrochloric acid, where the end point of the titration is a point that phenolphthalein as an indicator becomes red. In addition, V2 is titer of 0.1N sodium hydroxide (ml) in the titration of a sample solution prepared by adding sodium hydroxide to the above titrated solution to a final concentration of 0.5N to make strong alkaline, and then heating at 40° C. for 20 minutes under stirring to make fully de-methylesterification, and then adding hydrochloric acid at the same amount of sodium hydroxide used in methylesterification, where titration is carried out in the same manner of V1.

### (Quantitation Method of Acetic Acid Ester)

A degree of acetic acid esterification is calculated as acetic acid ester content relative to water-soluble soybean polysaccharide according to the following formula after quantitation of free acetic acid generated by hydrolyzing an acetic acid esterified water-soluble soybean polysaccharide:

Acetic acid content=1.4×V2−V1

In this formula, V1 is an acetic acid content of a sample solution measured by ion chromatography where the sample solution is prepared by passing 5 ml of 0.3 wt % solution of water-soluble soybean polysaccharide as a sample through a filter of molecular weight cut off 10,000. In addition, V2 is an acetic acid content of a sample solution in the same manner of V1 except that the sample solution is prepared by adding 1 ml of 0.5N sodium hydroxide to 5 ml of 0.3 wt % of the same sample, and then carrying out ester decomposition at 40° C. for 20 minutes, and then adding same amount, 1 ml of 0.5N hydrochloric acid to neutralization, and then passing the neutralized solution through a filter of molecular weight cut off 10,000, and then measuring the acetic acid content. From the above formula, an acetic acid content in a water-soluble soybean polysaccharide is calculated.

An ion chromatography is carried out by using Compact IC 861 (manufactured by Metrohm Japan Ltd.); column: Shodex RS Pak KC-811 (φ 8 mm×300 mm) at 50° C.; eluting solution: 1 mM perchloric acid (flow rate 1 ml/min); detector: conductivity meter; and standard substance: sodium acetate.

### (Intended Purpose)

A water-soluble soybean polysaccharide of the present invention provides emulsifying capacity and emulsion-stabilizing property with lower amount, which has been difficult to achieve in a conventional technology. In addition, the water-soluble soybean polysaccharide can provide sufficient emulsifying capacity with a gentle stirring and without using an apparatus for applying high shearing force, such as high-pressure homogenizer, and has less effect on taste, and therefore, can be used as an emulsifier for preparing more various kind of oil-in-water (O/W type) emulsion or W/O/W type emulsion than in the past.

In addition, since the water-soluble soybean polysaccharide provides excellent dispersion stability of emulsion, it can be used as an emulsifier for preparing oil-in-water emulsion or W/O/W type emulsion in the preparation of drug medicine, quasi-drug, cosmetics or the like.

More specifically, in a food industry, a water-soluble soybean polysaccharide of the present invention can be used as an emulsifier for emulsification of food; such as beverages such as soft drink, milk beverage, soy beverage, fruit juice drink, tea, sports drink, powdered drink and alcoholic drink; sweets such as candy, gummy candy, jelly and chewing gum; frozen desserts such as ice cream; dressing; mayonnaise; baked products; processed seafood; processed meat and retort food, oil-based flavor, and oil-based color. In addition, it is also effective to add to the above described food as an emulsified flavor described later.

In a non-food industry, the water-soluble soybean polysaccharide of the present invention can be used as an emulsifier for emulsification of hair washing products such as shampoo and hair conditioner; hair cosmetics such as hair treatment, hair lotion and hair wax; various skin cosmetics such as lip rouge, face lotion, cleansing cream, shaving cream, face-wash, skin milk, hand soap, foundation, moisturizing essence, body-wash and makeup remover; drug medicine; quasi-drug; chemical products; feedstuff; agrichemical; print or the like.

The above described water-soluble soybean polysaccharide can be used as an emulsifier with remaining a form of solution or after drying and powderization. It can also be used as an emulsified product including other carrier and additives. In this case, the carrier and the additives can be chosen depending on the kind and usage of the product in which the emulsifier is used. For example, the water-soluble soybean polysaccharide can be used with mixing with multiple alcohol such as glycerin; dextrin; or saccharide such as lactose.

### (Emulsified Flavor)

As an especially advantageous usage of the water-soluble soybean polysaccharide of the present invention includes use for an emulsified flavor. The emulsified flavor is an oil-in-water (O/W type) emulsion prepared by emulsifying a phase composed of hydrophobic material by using an emulsifier such as gum Arabic, where the hydrophobic material is prepared by dissolving a refined essential oil or prepared flavor into plant oil.

For example, in a case of using a water-soluble soybean polysaccharide of the present invention to an emulsified flavor, 0.2 to 5 parts by weight, preferably 0.5 to 3 parts by weight of the water-soluble soybean polysaccharide relative to 1 part by weight of included hydrophobic material (oil component) is added. In a case of general emulsified flavor which includes 20 wt % of oil phase, it corresponds to adding amount of 4 to 100 wt % of the water-soluble soybean polysaccharide. On the other hand, when the conventional gum Arabic is not added at a content of more than 12 wt % to the above oil phase (more than 0.6 part by weight relative to 1 part by weight of oil content), emulsion particle diameter tends to be increased over time due to insufficient emulsifying capacity and long-term emulsion stability tends to be lost. That is, the present invention has remarkable effect in adding 0.2 part by weight or more and less than 0.6 part by weight, preferably 0.5 part by weight or more and less than 0.6 part by weight of the water-soluble soybean polysaccharide relative to 1 part by weight of oil content, as compared with gum Arabic. In addition, in the case of emulsified flavor for alcohol beverage described later, the present invention has remarkable effect as compared with gum Arabic even if the emulsifier is used at higher concentration.

Various apparatuses can be used for stabilizing emulsification, and it can be chosen depending on the desired particle diameter and viscosity etc. of the water-soluble soybean polysaccharide. The purpose can be achieved by using only a mixer such as colloid mill, dispersion mill, Homomixer and propeller stirrer, as well as using emulsification equipment such as high-pressure homogenizer and ultrasonic homogenizer. For example, fine and uniform oil-in-water emulsion can be obtained with gently stirring without using a high-pressure homogenizer by using the water-soluble soybean polysaccharide while conventional emulsification using gum Arabic requires pre-emulsification with Homomixer to make a dispersion particle diameter is reduced on some level before homogenization treatment with a high-pressure homogenizer which is an expensive device. That is, it can lead to labor saving.

An emulsified hydrophobic material in the emulsified flavor is not particularly limited, and a material generally used as a dispersing phase can be used. Specific Example of an oil-based flavor includes essential oil from citrus such as orange, grapefruit, summer mandarin orange (Citrus natsudaidai), bergamot, lime, lemon and Yuzu (Citrus junos); essential oil from plant such as flower oil, spearmint oil and peppermint oil; essential oil or oleoresin from spice such as onion, garlic, cardamom, cumin, clove, ginger, celery, nutmeg, basil, parsley, paprika, black pepper, rosemary and laurel; oil-based extract such as kola-nut extract, coffee extract, cocoa extract, tea extract, spice extract and vanilla extract, or oleoresin thereof; flavor material such as eugenol, geraniol, acetic acid, diacetyl, citral, vanillin, ethyl propionate, menthol, butyric acid and limonene; synthetic flavor compound; blended oil-based flavor composition; and any combination thereof.

Animal oil and plant oil to be added to the above oil-based flavor include, for example, olive oil, cacao butter, corn oil, sesame oil, wheat germ oil, rice oil, rice bran oil, safflower oil, soybean oil, camellia oil, rapeseed oil, palm oil, sunflower seed oil, cottonseed oil, coconut oil, peanut oil, beef tallow, lard, chicken fat, fish oil, butter and the like. A medium-chain saturated fatty acid triglyceride includes a triglyceride having 6 to 12 carbon atoms and used as processed cooking oil, for example, caproic acid triglyceride, caprylic acid triglyceride, capric acid triglyceride, lauric acid triglyceride and the like. An oil-based color includes, for example, an oil-soluble natural color such as annatto color, chlorophyl, β-carotene and paprika color, and the like. A fat-soluble vitamin includes, for example, liver oil, vitamin A, vitamin A oil, vitamin B2 tetrabutyrate, vitamin D3, natural vitamin E mixture and the like. A natural resin includes, for example, plant resin such as elemi, ester gum, copal, dammar and rosin.

These edible oil-based materials can be used alone or as a mixture of two or more kinds.

The above explained emulsified flavor is suitable for, especially, alcohol beverage. The alcohol beverage refers to a beverage including 3 to 50% by volume, preferably 20 to 35% by volume, of ethyl alcohol. A conventional emulsified flavor has insufficient function for preventing oil float while maintaining turbidity by emulsified particle in storing of the alcohol beverage, especially in the system of including 20% by volume or more of ethyl alcohol. An emulsified flavor prepared by using a water-soluble soybean polysaccharide of the present invention shows remarkable high emulsion-stabilizing property when it is used for alcohol beverage as compared with an emulsified flavor prepared by using gum Arabic which is generally used. That is, the emulsified flavor including the water-soluble soybean polysaccharide of the present invention is most suitable for an alcohol beverage.

### EXAMPLES

The present invention will be more specifically explained below by way of examples and comparative examples. The “%”, “parts” described below refers to “wt %”, “parts by weight” unless otherwise specified.

### Example 1

### De-Methylesterification Treatment (pH 12), Acetic Acid Esterification Treatment (20%)

Okara generated in the production of soybean protein isolate was used as a raw material. Water was added to the okara, and pH was adjusted to 5 with hydrochloric acid. Then, extraction of water-soluble soybean polysaccharide was carried out with a pressure vessel at 125° C. for 2 hours. After the extraction, centrifugation (5,000×g, 10 minutes) was carried out to separate a supernatant including most of water-soluble soybean polysaccharide and a precipitate. Sodium hydroxide was added to the supernatant to adjust pH to 12. Then, de-methylesterification treatment was carried out by heating at 60° C. for 60 minutes. Precipitate generated by the heating was removed, and then hydrochloric acid was added to adjust pH to 5.0, and then the generated precipitate was removed. Then, demineralization with electrodialysis was carried out to concentrate an aqueous solution of the water-soluble soybean polysaccharide to 10Bx, and then pH was adjusted to 8.5 with sodium hydroxide solution.

Anhydrous acetic acid was added little by little to become 20 wt % relative to dry matter for 30 minutes while keeping 40° C. and keeping pH of 8.5 with a pH automatic titration device, and then reacting for 1 hours while keeping pH of 8.5. After the reaction, the solution was subjected to solvent precipitation with ethanol, washing, and then drying at room temperature to obtain “water-soluble soybean polysaccharide A”. Methyl esterification degree and content of esterified acetic acid of the obtained water-soluble soybean polysaccharide were determined by the above described method.

### Example 2

### De-Methylesterification Treatment (pH 11), Acetic Acid Esterification Treatment (10%)

An extraction of water-soluble soybean polysaccharide, and de-methylesterification treatment and acetic acid esterification treatment were carried out in the same manner as Example 1 except that the de-methylesterification treatment was carried out at pH 11 and that 10 wt % of anhydrous acetic acid relative to dry matter was added in the acetic acid esterification treatment. Analysis of the obtained “water-soluble soybean polysaccharide B” was carried out in the same manner as Example 1.

### Example 3

### De-Methylesterification Treatment (pH 10), Acetic Acid Esterification Treatment (5%)

An extraction of water-soluble soybean polysaccharide, and de-methylesterification treatment and acetic acid esterification treatment were carried out in the same manner as Example 1 except that the de-methylesterification treatment was carried out at pH 10 and that 5 wt % of anhydrous acetic acid relative to dry matter was added in the acetic acid esterification treatment. Analysis of the obtained “water-soluble soybean polysaccharide C” was carried out in the same manner as Example 1.

### Comparative Example 1

### Without De-Methylesterification Treatment, without Acetic Acid Esterification Treatment

A water-soluble soybean polysaccharide was extracted in the same manner as Example 1. Analysis of the obtained “water-soluble soybean polysaccharide D” without de-methylesterification treatment and without acetic acid esterification treatment was carried out in the same manner as Example 1.

### Comparative Example 2

### De-Methylesterification Treatment (pH 12), without Acetic Acid Esterification Treatment

An extraction of water-soluble soybean polysaccharide, and de-methylesterification treatment were carried out in the same manner as Example 1. Analysis of the obtained “water-soluble soybean polysaccharide E” without acetic acid esterification treatment was carried out in the same manner as Example 1.

### Comparative Example 3

### Without De-Methylesterification Treatment, Acetic Acid Esterification Treatment (20%)

An extraction of water-soluble soybean polysaccharide, and acetic acid esterification treatment were carried out in the same manner as Example 1 except that 20 wt % of anhydrous acetic acid relative to dry matter was added in the acetic acid esterification treatment. Analysis of the obtained “water-soluble soybean polysaccharide F” without de-methylesterification treatment was carried out in the same manner as Example 1.

Results of quality evaluation of the water-soluble soybean polysaccharides obtained in Example 1, Example 2, Example 3, Comparative Example 1, Comparative Example 2 and Comparative Example 3 were shown in Table 1.

TABLE 1
Analysis values of each water-soluble soybean polysaccharides

| | Example | Example | Example | Comparative | Comparative | Comparative |
|---|---|---|---|---|---|---|
| | 1 | 2 | 3 | Example 1 | Example 2 | Example 3 |
| Water-soluble | A | B | C | D | E | F |
| soybean | | | | | | |
| polysaccharide | | | | | | |
| De- | 12 | 11 | 10 | — | 12 | — |
| methylesterification | | | | | | |
| treatment (pH) | | | | | | |
| Acetic acid | 20% | 10% | 5% | — | — | 20% |
| esterification | | | | | | |
| treatment | | | | | | |
| (anhydrous acetic | | | | | | |
| acid %) | | | | | | |
| Methyl | 8.0 | 15.0 | 25.0 | 48.8 | 6.8 | 56.0 |
| esterification | | | | | | |
| degree (%) | | | | | | |
| Content of | 7.9 | 4.0 | 1.5 | 1.6 | 0 | 8.8 |
| esterified acetic | | | | | | |
| acid | | | | | | |

From the above results, it was clearly found that residual methyl ester tended to be lower when starting pH of de-methylesterification treatment was higher, and that content of esterified acetic acid tended to be higher when an adding amount of anhydrous acetic acid was higher in the acetic acid esterification treatment. In addition, acetic acid which was originally contained as ester in the water-soluble soybean polysaccharide was detected because neither de-methylesterification treatment nor acetic acid esterification treatment were carried out in Comparative Example 1. On the other hand, it was thought that acetic acid ester was cleaved by de-methylesterification treatment in Comparative Example 2.

### Application Example A

### Preparation of Emulsified Composition

Each emulsified compositions were prepared according to the following procedure by using the water-soluble soybean polysaccharides A to F obtained by the above described process, respectively.

To 100 g of distilled water, 16.0 g of glycerin was added and each 21.4 g of the water-soluble soybean polysaccharides A to F was dissolved while stirring, and then pH of the each solutions was adjusted to 4.0 by 50% solution of citric acid. And then, 40 g of mixture including lemon oil, MCT (medium-chain saturated fatty acid triglyceride) and Sucrose diacetate hexaisobutyrate (sucrose acetate isobutyrate) at a weight ratio of 2:3:5 (specific gravity d 1.010) was added to the each aqueous solutions of water-soluble soybean polysaccharides A to F, and then water was added to adjust a total amount of 200 g, and then the solutions were stored at 45° C. The stored solution was pre-stirred with a homogenizer, “Polytron” (manufactured by KINEMATICA AG) at 10,000 rpm for 10 minutes, and then homogenized twice with a high pressure homogenizer, “MINI-LAB type 8.30H” (manufactured by RANNIE) at a pressure of 150 kgf/cm2 (14.71 MPa) to obtain emulsified compositions (Application Examples 1 to 3, Comparative Application Examples 1 to 3). Each composition was stored in a refrigerator. Particle diameter of the composition was measured with a laser diffraction particle size analyzer, “SALD2000A” (manufactured by Shimadzu Corporation) immediate after and 7 days after the preparation. The results were shown in table 2.

TABLE 2
Particle diameter of each emulsion after storage

| | Appl. | Appl. | Appl. | Comparative | Comparative | Comparative |
|---|---|---|---|---|---|---|
| | Example | Example | Example | Appl. | Appl. | Appl. |
| | 1 | 2 | 3 | Example 1 | Example 2 | Example 3 |
| Water-soluble | A | B | C | D | E | F |
| soybean | | | | | | |
| polysaccharide | | | | | | |
| Amount of water- | 10.7 | ← | ← | ← | ← | ← |
| soluble soybean | | | | | | |
| polysaccharide in | | | | | | |
| the system (%) | | | | | | |
| Amount of water- | 0.535 | ← | ← | ← | ← | ← |
| soluble soybean | | | | | | |
| polysaccharide | | | | | | |
| relative to 1 part | | | | | | |
| of the oil phase | | | | | | |
| (hydrophobic | | | | | | |
| material) (part) | | | | | | |
| Median particle | 0.417 | 0.440 | 0.534 | 0.638 | 0.627 | 0.573 |
| diameter immediate | | | | | | |
| after the | | | | | | |
| preparation (μm) | | | | | | |
| Median particle | 0.415 | 0.441 | 0.541 | 0.679 | 0.632 | 0.577 |
| diameter 7 days | | | | | | |
| after the | | | | | | |
| preparation (μm) | | | | | | |

It was clear from the above table 2 that, in the system of emulsion including 20 wt % of oil phase and 10.7 wt % of additive amount of water-soluble soybean polysaccharide, Application Examples 1 to 3 which were subjected to both de-methylesterification treatment and acetic acid esterification treatment showed 0.6 μm or less of particle diameter and had superior emulsifying capacity as compared with the conventional water-soluble soybean polysaccharide used in Comparative Application Examples 1 to 2 (Water-soluble soybean polysaccharides D, E). Especially, Application Example 1 and Application Example 2 showed excellent particle diameter, 0.5 μm or less, and uniform particle size distribution, and showed good stability of the emulsified compositions even after 7 days storage in a refrigerator. On the other hand, Comparative Application Examples 1 to 2 (water-soluble soybean polysaccharides D, E) as conventional water-soluble soybean polysaccharide, and Comparative Application Example 3 which was subjected to only acetic acid esterification treatment showed about 0.7 μm of particle diameter which was larger than that of Application Examples 1 to 3 in the emulsifying capacity. In addition, change of the particle diameter after 7 days storage in a refrigerator was small. In this case, Comparative Application Examples 2 and 3 showed good stability.

In the case of gum Arabic, it is necessary to add generally high concentration, 12 wt % or more in a system of 20 wt % of oil phase in order to obtain stable emulsion state. On the other hand, it was found that addition of 10.7 wt % of the water-soluble soybean polysaccharide could provide particle diameter small, excellent emulsifying capacity and high emulsion-stabilizing property.

### Application Example B

### Preparation of Concentrated Syrup

Evaluation of the emulsified composition of Application Examples 1 to 3 and Comparative Application Examples 1 to 3 was carried out in a condition of high acid degree and alcohol concentration (concentrated syrup of alcohol beverage). To 60 g of water, 1.14 g of citric acid, 0.33 g of sodium citrate and 11.8 g of isomerized sugar were dissolved, and then 21 ml of ethanol was added, and then total volume of the syrup was adjusted to 100 ml with water to prepare concentrated syrup. To the concentrated syrup, 0.3 g of the emulsified composition of Application Examples 1 to 3 or Comparative Application Examples 1 to 3 was added, and the prepared syrup was stored at room temperature. Oil float, turbidity (OD 680) and particle diameter (laser diffraction particle size analyzer) of the prepared syrup was measured immediately after or 3 days after the preparation (Application Examples 4 to 6, and Comparative Application Examples of 4 to 6). The results were shown in table 3.

TABLE 3
Particle diameter of concentrated syrup after storage

| | | | | Particle |
|---|---|---|---|---|
| | | Oil float | OD680 | diameter (μm) |
| Application | Immediately | − | 0.095 | 0.413 |
| Example 4 | after | | | |
| | preparation | | | |
| | After 3 days | − | 0.104 | 0.413 |
| Application | Immediately | − | 0.120 | 0.440 |
| Example 5 | after | | | |
| | preparation | | | |
| | After 3 days | − | 0.130 | 0.443 |
| Application | Immediately | − | 0.335 | 0.567 |
| Example 6 | after | | | |
| | preparation | | | |
| | After 3 days | + | 0.321 | 0.570 |
| Comparative | Immediately | − | 0.493 | 0.644 |
| Application | after | | | |
| Example 4 | preparation | | | |
| | After 3 days | ++ | 0.498 | 0.665 |
| Comparative | Immediately | − | 0.484 | 0.637 |
| Application | after | | | |
| Example 5 | preparation | | | |
| | After 3 days | ++ | 0.488 | 0.654 |
| Comparative | Immediately | − | 0.503 | 0.577 |
| Application | after | | | |
| Example 6 | preparation | | | |
| | After 3 days | ++ | 0.505 | 0.603 |

−: No oil float,
+: Oil float was slightly observed,
++: A lot of oil float

It was clear from the above table 3 that all of Application Examples 4 to 6 showed 0.6 μm or less of particle diameter and had superior emulsifying capacity as compared with the conventional water-soluble soybean polysaccharide used in Comparative Application Examples 4 to 5 (Water-soluble soybean polysaccharides D, E). Especially, Application Examples 4 to 5 (Water-soluble soybean polysaccharides A, B) showed excellent particle diameter, 0.5 μm or less. In addition, after 3 days storage, Application Examples 4 to 5 showed good stability of the emulsified composition without generating oil float. In addition, oil float was slightly observed in Application Example 6. On the other hand, Comparative Application Examples 4 to 5 as conventional water-soluble soybean polysaccharide, and Comparative Application Example 6 which was subjected to only acetic acid esterification treatment showed 0.7 μm or less of particle diameter. Although this particle diameter was smaller than 0.8 μm, which is typical particle diameter of gum Arabic, oil float was remarkably observed after 1 day storage at room temperature in these Comparative Application Examples. Therefore, these Comparative Application Examples lacked emulsion stability.

### Application Example C

### Preparation of Emulsified Composition with Low Shearing Force (I)

The emulsified compositions obtained in Application Examples 1 to 3 were subjected to homogenization with high pressure homogenizer at 150 kgf/cm2 (14.71 MPa) twice to make the particle diameter small to 0.4 to 0.7 μm. Here, various emulsified compositions were prepared by using the water-soluble soybean polysaccharides A and D obtained in Example 1 and Comparative Example 1 without using high pressure homogenizer according to the following procedure.

To 48 g of distilled water, 8 g of glycerin was added, and then 16 g of the water-soluble soybean polysaccharide A or the water-soluble soybean polysaccharide D was dissolved with stirring, and then the obtained solution was adjusted to pH 4.0 with 50 wt % citric acid solution. And then, 16 g of mixture including lemon oil, MCT (medium-chain saturated fatty acid triglyceride) and Sucrose diacetate hexaisobutyrate (sucrose acetate isobutyrate) at a weight ratio of 2:3:5 (specific gravity d 1.010) was added to the aqueous solution of water-soluble soybean polysaccharide A or D, and then water was added to adjust a total amount of 100 g, and then the solution was stored in ice. The stored solution was stirred with a stirrer, “Polytron” (manufactured by KINEMATICA AG) at 7,500 rpm for 60 minutes to obtain emulsified composition (Application Example 7, Comparative Application Example 7). During the preparation, minimum amount of sample required to measure a particle diameter was recovered in order to confirm the emulsified state after 20 minutes of the stirring.

In addition, the emulsified composition was obtained in the same manner as the above except that rotation rate was changed from 7,500 rpm to 5,000 rpm (Application Example 8, Comparative Application Example 8). Further, the emulsified composition was obtained in the same manner as the above except that rotation rate was changed from 7,500 rpm to 2,500 rpm (Application Example 9, Comparative Application Example 9).

For the emulsified compositions of Application Examples 7 to 9 and Comparative Application Examples 7 to 9, particle diameter of the composition was measured with a laser diffraction particle size analyzer, “SALD2000A” (manufactured by Shimadzu Corporation) immediate after and after 7 days storage in a refrigerator. The results were shown in table 4.

TABLE 4
Particle diameter of emulsion prepared with low shearing force after storage

| | | | Comparative | | Comparative | | Comparative | |
|---|---|---|---|---|---|---|---|---|
| | | Application | Application | Application | Application | Application | Application | |
| | | Example 7 | Example 7 | Example 8 | Example 8 | Example 9 | Example 9 | |
| Water-soluble soybean polysaccharide | | A | D | A | D | A | D | |
| Rotation rate for stirring | | 7,500 | 7,500 | 5,000 | 5,000 | 2,500 | 2,500 | (rpm) |
| After 20 min. stirring | Immediately after | 1.36 | 1.55 | 1.60 | 2.02 | 2.79 | 3.68 | (μm) |
| After 60 min. stirring | preparation | 0.90 | 1.21 | 1.23 | 1.60 | 1.75 | 2.64 | |
| After 20 min. stirring | After 7 days | 1.37 | 1.55 | 1.64 | 1.99 | 2.77 | 3.75 | |
| After 60 min. stirring | | 0.93 | 1.21 | 1.25 | 1.76 | 1.70 | 2.66 | |

As shown in the above results, the water-soluble soybean polysaccharide A showed higher emulsifying capacity than the water-soluble soybean polysaccharide D at the same rotation rate and stirring time, and could maintain particle diameter of, 1.5 μm or less at 7,500 rpm, 2 μm or less at 5,000 rpm, and 3 μm or less at 2,500 rpm after 7 days storage in the refrigerator even if the preparation was carried out with low shearing force and without using a high pressure homogenizer. In addition, in the case of 60 minutes stirring, particle diameter of, 1 μm or less at 7,500 rpm, 1.5 μm or less at 5,000 rpm, and 2 furl or less at 2,500 rpm could be maintained after 7 days storage in the refrigerator.

### Application Example D

### Preparation of Emulsified Composition with Low Shearing Force (II)

It was found that the emulsified compositions obtained in Application Examples 7 to 9 provided 2 μm or less of particle diameter at a comparatively low rotation rate such as 2,500 rpm to 7,500 rpm. Further, particle diameter provided by slower stirring was confirmed.

To 48 g of distilled water, 8 g of glycerin was added, and then 16 g of the water-soluble soybean polysaccharide A, the water-soluble soybean polysaccharide D or gum Arabic (Arabic Cole SS, manufactured by San-ei Yakuhin Boeki Co., Ltd.) was dissolved with stirring, and then the obtained solution was adjusted to pH 4.0 with 50 wt % citric acid solution. And then, 16 g of mixture including lemon oil, MCT (medium-chain saturated fatty acid triglyceride) and Sucrose diacetate hexaisobutyrate (sucrose acetate isobutyrate) at a weight ratio of 2:3:5 (specific gravity d 1.010) was added to each of the aqueous solution, and then water was added to adjust a total amount of 100 g, and then the solution was stored in ice. The stored solution was stirred with a stirrer, “MAZELA Z” (manufactured by Tokyo Rikakikai Co., LTD.) at 1,000 rpm for 60 minutes to obtain emulsified composition (Application Example 10, Comparative Application Examples 10, 11). During the preparation, minimum amount of sample required to measure a particle diameter was recovered in order to confirm the emulsified state after 20 minutes of the stirring.

In addition, the emulsified composition was obtained in the same manner as the above except that rotation rate was changed from 1,000 rpm to 500 rpm (Application Example 11, Comparative Application Examples 12, 13).

For the emulsified compositions of Application Examples 10 to 11 and Comparative Application Examples 11 to 13, particle diameter of the composition was measured with a laser diffraction particle size analyzer, “SALD2000A” (manufactured by Shimadzu Corporation) immediate after the preparation and after 7 days storage in a refrigerator. The results were shown in table 5.

TABLE 5
Particle diameter of emulsion prepared with low shearing force after storage

| | | | Comparative | Comparative | | Comparative | Comparative | |
|---|---|---|---|---|---|---|---|---|
| | | Application | Application | Aplication | Application | Application | Application | |
| | | Example 10 | Example 10 | Example 11 | Example 11 | Example 12 | Example 13 | |
| Water-soluble soybean polysaccharide | | A | D | — | A | D | — | |
| Gum Arabic | | — | — | SS | — | — | SS | |
| Rotation rate for stirring | | 1,000 | 1,000 | 1,000 | 500 | 500 | 500 | (rpm) |
| After 20 min. stirring | Immediately after | 3.93 | 5.34 | 8.64 | 6.25 | 8.13 | 26.63 | (μm) |
| After 60 min. stirring | preparation | 3.60 | 4.08 | 4.77 | 5.40 | 7.52 | 19.76 | |
| After 20 min. stirring | After 7 days | 3.94 | 5.82 | 9.85 | 6.57 | 8.43 | 27.11 | |
| After 60 min. stirring | | 3.62 | 3.99 | 6.19 | 5.66 | 7.59 | 19.94 | |

As shown in the above results, the water-soluble soybean polysaccharide A showed higher emulsifying capacity than the water-soluble soybean polysaccharide D at the same rotation rate and stirring time, and could maintain particle diameter of, 4 μm or less at 1,000 rpm, and 7 μm or less at 500 rpm after 7 days storage in the refrigerator even if the preparation was carried out with low shearing force and without using a high pressure homogenizer. In addition, gum Arabic provided particle diameter of, about 10 μm at 1,000 rpm, and about 27 μm at 500 rpm, had weak emulsifying capacity and could not provide small particle diameter by low shearing force only by stirring.

Further, the water-soluble soybean polysaccharide A could maintain particle diameter of, 4 μm or less at 1,000 rpm, and 6 μm or less at 500 rpm after 7 days storage in the refrigerator in the case of 60 minutes stirring. In addition, gum Arabic could provide about 6 μm at 1,000 rpm, but required long time stirring. Further, it could provide about 20 μm at 500 rpm even if stirring was carried out long time. It had weak emulsifying capacity. That is, gum Arabic could not provide small particle diameter by low shearing force stirring.

### Application Example E

### Preparation of Clear Shampoo

Shampoo was prepared by preparing oil-in-water emulsion by mixing and stirring the water-soluble soybean polysaccharide A, the water-soluble soybean polysaccharide D or gum Arabic used in the above Application Example C according to the formulation shown in table 6 (Application Example 13, Comparative Application Examples 14, 15).

TABLE 6
Formulation of shampoo

| | | Comparative | Comparative |
|---|---|---|---|
| | Application | Application | Application |
| Components (wt %) | Example 13 | Example 14 | Example 15 |
| 2-lauryl-N-carboxymethyl- | 10 | ← | ← |
| N-hydroxyethyl | | | |
| imidazolinium betaine | | | |
| Triethanolamine laurate | 5 | ← | ← |
| Coconut fatty acid | 5 | ← | ← |
| diethanolamide | | | |
| Water-soluble soybean | 2.5 | — | — |
| polysaccharide A | | | |
| Water-soluble soybean | — | 2.5 | — |
| polysaccharide D | | | |
| Gum Arabic | — | — | 2.5 |
| Edetate disodium | 0.1 | ← | ← |
| Flavor, color, antiseptic | Appropriate | ← | ← |
| | amount | | |
| Purified water | Residual | ← | ← |

The shampoo of Application Example 13 including the water-soluble soybean polysaccharide A provided remarkably improved quality in the smoothness and good hydration of hair. On the other hand, the shampoo of Comparative Application Example 15 including conventional gum Arabic provided insufficient texture that was stickiness after applying to hair or skin, and that was tightness and stiffness after drying on hair or skin. The shampoo of Comparative Application Example 14 including the water-soluble soybean polysaccharide D also provided smoothness and good hydration of hair, but these were less than the Application Example 13.

### Application Example F

### Preparation of Skin Cosmetic

An oil-in-water emulsion prepared by mixing and stirring 25 parts of the water-soluble soybean polysaccharide A, 1 part of hydroxypropylcellulose and 74 parts of water was adjusted to pH 6 with sodium hydroxide solution to provide a skin cosmetic (Application Example 12).

### INDUSTRIAL APPLICABILITY

Emulsified particles can be stabilized by using a water-soluble soybean polysaccharide of the present invention with smaller additive amount than conventional gum Arabic. In addition, an emulsified flavor having high tolerance in alcoholic drink can be prepared. Further, particle diameter of fluorescent paint or toner can be stabilized to 1 to 3 μm only by pre-emulsifying treatment and stabilized particle diameter after shaking can be kept without using high pressure homogenizer.

Moreover, according to the present invention, production cost can be suppressed and efficiency of the production process can be improved because an existing water-soluble soybean polysaccharide can be dissolved as it is, and the de-methylesterification treatment and the acetic acid esterification treatment can be carried out easily in a general tank which is used in the industry, and the yield is also good.

1. A water-soluble soybean polysaccharide comprising 30% or less of methyl ester content relative to uronic acid and 1 wt % or more of acetic acid ester content relative to the water-soluble soybean polysaccharide in terms of free acetic acid.

2. A process for producing the water-soluble soybean polysaccharide according to claim 1, comprising subjecting acetic acid esterification treatment to water-soluble soybean polysaccharide which is subjected to de-methylesterification treatment.

3. An emulsifier comprising the water-soluble soybean polysaccharide according to claim 1 as an active ingredient.

4. An oil-in-water emulsion obtained by using the emulsifier according to claim 3 to stabilizing dispersion of hydrophobic material to hydrophilic solvent.

5. The oil-in-water emulsion according to claim 4, wherein 0.3 part by weight or more and 7 parts by weight or less of the emulsifier is used to 1 part by weight of hydrophobic material.

6. A use of the water-soluble soybean polysaccharide according to claim 1 for preparing an emulsifier.

7. A use of the water-soluble soybean polysaccharide according to claim 1 for preparing an emulsion.
